(19) United States
(12) Patent Application Publication
(10) Pub. No.: US 2004/0171163 A1
(43) Pub. Date: Sep. 2, 2004

(54) Electrical conductive containment system

(76) Inventors: Peter A. Lopez, Bridgeport, CT (US); Peter A. Lopez, Bridgeport, CT (US); Kris S. Buchanan, Fort Collins, CO (US)

Correspondence Address: SANTANGELO LAW OFFICES, P.C., 125 SOUTH HOWES, THIRD FLOOR, FORT COLLINS, CO, 80521, US

(21) Appl. No.: 10/450,931

(22) PCT Filed: Dec. 14, 2001
(86) PCT No.: PCT/US01/48185

(30) Foreign Application Priority Data
Dec. 15, 2000 (US) 60256070

(51) Int. Cl. G01N 33/48

(52) U.S. Cl. 436/63; 422/102; 422/73

(57) ABSTRACT

Specifically, electrically conductive droplet collection containers (12) that enhance the collection and retention of electrostatically deflected droplets (13) containing particles (27) formed in a fluid stream (21) in applications such as flow cytometry.

This application claims the benefit of United States Provisional Patent Application No. 60/256,070, filed Dec. 15, 2000, hereby incorporated by reference.

### I. TECHNICAL FIELD

Generally, an electrically conductive containment system that provides containers, container securement or container movement components, or the like having sufficient electrical conductivity to maintain a neutral charge when electrically coupled to ground or maintain a selected charge to all or a portion of the containment system. Specifically, a flow cytometer system providing electrically conductive collection containers to enhance the collection and retention of electrostatically deflected droplets by preferentially charging the droplet collection containers or maintaining a neutral charge.

### II. BACKGROUND

As particles charged with the same electrical polarity are accumulated in a collection container, the net result can be that the collection container itself assumes the electrical polarity of the particles being collected therein. Once this electrical charge of the collection container becomes established, incoming particles of the same polarity can be deflected away from the collection container since there is an electrostatic force operating to separate charges of like nature. As such, an electrically charged collection container can cause the loss of particles that normally would be collected within such collection container.

Moreover, the speed of particle or droplet forming and electrostatic particle separation technology has increased dramatically in the past few years. Droplets can be formed in fluid streams at rates of 10,000 droplets per second, 20,000 droplets per second, even as many as 80,000 droplets per second. Moreover, particle or material collection containers, such as those having wells geometrically arranged in columns or rows, such as microtiter plates, can have a large electrostatically charged surface area relative to the target area of the container aperture(s) making small changes in the trajectory of the particles more probable and more likely to cause the loss of particles as described above.

Particle sorters of the type relying upon electrostatic separation of particles such as those described in U.S. Pat. Nos. 3,380,584; 3,710,933; 3,826,364; 4,148,718; 4,230,558; and 4,318,480, each hereby incorporated by reference, inasmuch as they rely upon an electrostatic field for separating and sorting particles, provide an example of a technology susceptible to the above-described problem wherein the collection containers become charged as particles of a specific polarity are accumulated.

The instant invention addresses the problem of the electrical charge building up on the components that make up the various types material collection systems.

### III. DISCLOSURE OF THE INVENTION

Accordingly, a broad object of embodiments of the invention can be to provide collection containers for the collection of droplets or particles that are electrically neutral and therefore less likely to cause loss of electrostatically deflected particles.

Another significant object of embodiments of the invention can be to provide electrically conductive containers that can be connected to ground. One aspect of the object can be to provide electrically conductive containers having a plurality of wells in configurations typically used to screen or collect a large numbers of samples, such as microtiter plates having 12, 24, 48, or 96 wells.

Another significant object of embodiments of the invention can be to provide securement elements for such electrically conductive containers to fix the orientation or position of the collection target or containment area.

Another significant object of embodiments of the invention can be to provide movement means to transfer such electrically conductive containers from a first zone to a second zone or from a first location to a second location, or between a plurality of locations, as desired.

Another significant object of embodiments of the invention can be to provide electrically conductive containers for use with flow cytometers. In certain embodiments of the invention, a portion or all of the electrically conductive container(s) can be preferentially charged to attract deflected particles of different charge to the collection target. In other embodiments of the invention, a portion of all of the electrically conductive container(s) can be maintained with a neutral charge.

Naturally further objects of the invention are disclosed throughout other areas of specification.

### IV. BRIEF DESCRIPTION OF THE DRAWINGS

FIG. 1 shows an embodiment of the invention comprising an electrically conductive material container.

FIG. 2 shows another embodiment of the invention comprising an electrically conductive multiple welled tray on a ground-connected surface.

FIG. 3 shows an embodiment of the invention comprising interlocking electrically conductive securement element to fix the orientation of an electrically conductive container.

FIG. 4 shows an embodiment of the invention comprising interlocking electrically conductive securement element to fix the orientation of an electrically conductive container and electrically conductive movement means to move the electrically conductive container to a location.

FIG. 5 shows an embodiment of the invention comprising an electrically conductive multiple welled tray and securement element with ground connect used in conjunction with an X-axis-Y-axis material movement means.

FIG. 6 shows an embodiment of the invention comprising an electrically conductive droplet collector.

FIG. 7 shows an embodiment of the invention comprising a flow cytometer system having electrically conductive droplet collection elements with ground connection.

FIG. 8 shows an enlarged view of an embodiment of the invention comprising a flow cytometer system having electrically conductive droplet collection elements with ground connection.

FIG. 9 shows an embodiment of the invention comprising electrically conductive droplet collection elements that have a portion or all of the containment area preferentially charged to attract particles to the containment target.

### V. MODE(S) FOR CARRYING OUT THE INVENTION

The invention involves various embodiments of a flow cytometer system and methods providing electrically conductive collection containers to enhance the collection and retention of electrostatically deflected droplets. More generally the invention involves material containment and material collection devices and techniques of containing and collecting materials into electrically conductive containers.

While particular embodiments of the invention are shown in the drawings and are described in detail along with the preferred embodiment of the invention, it should be understood that that the present disclosure of these embodiments of the invention is to be considered as exemplary of the principles of the invention and is not intended to limit the invention to those embodiments illustrated.

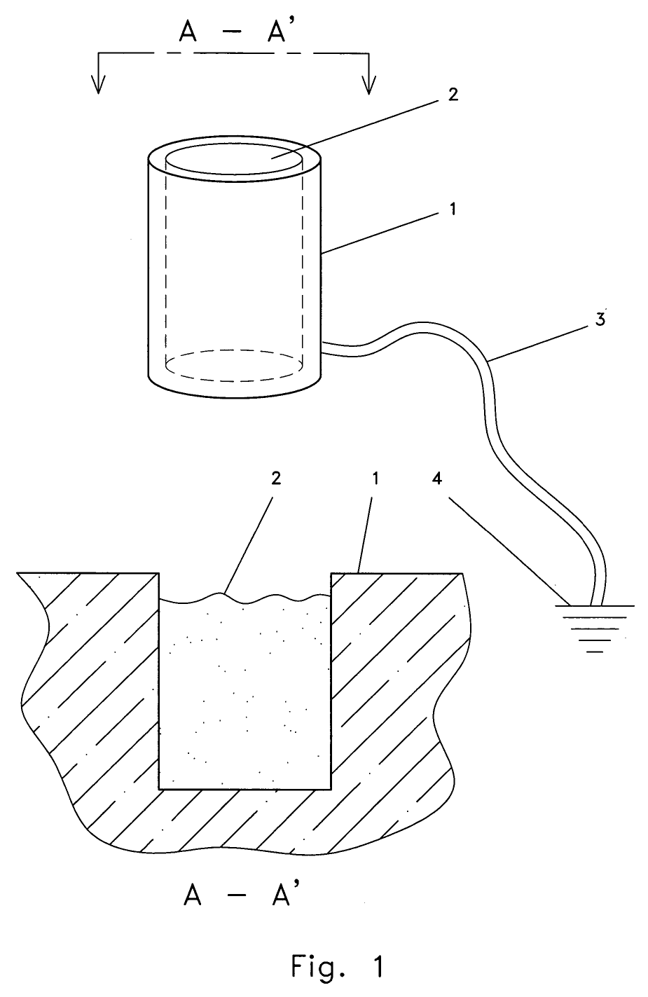

Now referring primarily to FIG. 1, the invention can comprise a material containment device having a container body (1) configured to have at least one material containment element (2) and further comprising an amount of electrically conductive material impregnated throughout the container body sufficient to allow the container body to maintain neutral charge when electrically connected to ground. In certain embodiments of the invention, the container body (1) can be configured from a substantially non-electrically conducting material. One example of such a non-electrically conducting material can be a plastic, such as, polystyrene, polycarbonate, polypropylene, polyacrylate, fluorocarbon, or similar polymers. In embodiments of the invention made from plastic, the plastic may be impregnated with metal particles, carbon, or polymerized to produce alternating single and double bonds between carbon atoms. In embodiments, where the carbon atoms have alternating single and double bonds between them, electrons must be further removed through oxidation or introduced through reduction of the polymer to create holes in the electronic structure of the polymer along which electrons can move--becoming electrically conductive. This process is often referred to as “doping” the material. The method of doping usefully employed in certain embodiments of the invention may be of a form as more fully described in the following references: Kungl. Vetenskapsakademien, The Royal Swedish Academy of Sciences, The Nobel Prize in Chemistry, 2000: Conductive Polymers (2000), hereby incorporated by reference herein.

The material containment element electrically conductive to ground can have a container body (1) can be formed, for example, as a substantially tubular configuration having at least one closed end as shown in FIG. 1, or can be a plurality of substantially tubular configurations geometrically arranged in relation to one another as would be useful or desired.

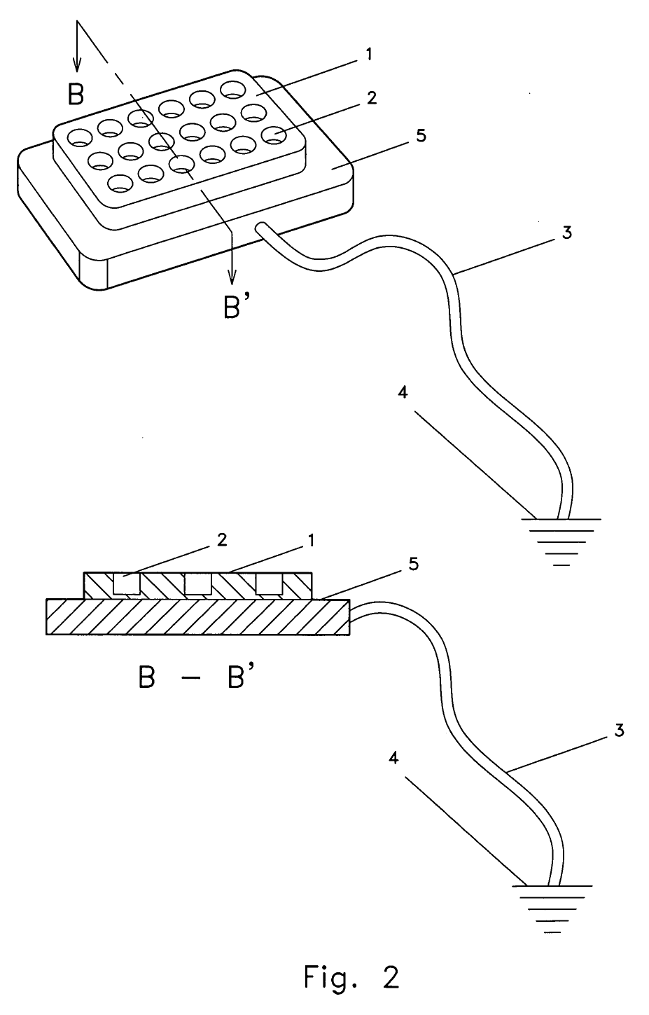

Now referring primarily to FIG. 2, one configuration can be a plurality of container bodies (1) geometrically arranged in a matrix of columns and rows. Often referred to as welled tray or microtiter plate, the number of material containment elements or wells can typically be 12, 24, 48, or 96. Naturally, the configuration of the container body (1) could be as desired to serve a particular material collection function or to mate or be compatible with a particular collector configuration, instrument configuration, or the like.

The invention further comprises a charge dissipation element that can comprise a ground connection (3) electrically coupled between the container body (1) and ground (4). In certain embodiments of the invention the ground connection can be established with a wire conductor between the electrically conductive container body (1) and the ground (4). In other embodiments of the invention the container body may be fixed to or movably interface with a grounded surface (5), such as that shown in FIG. 2.

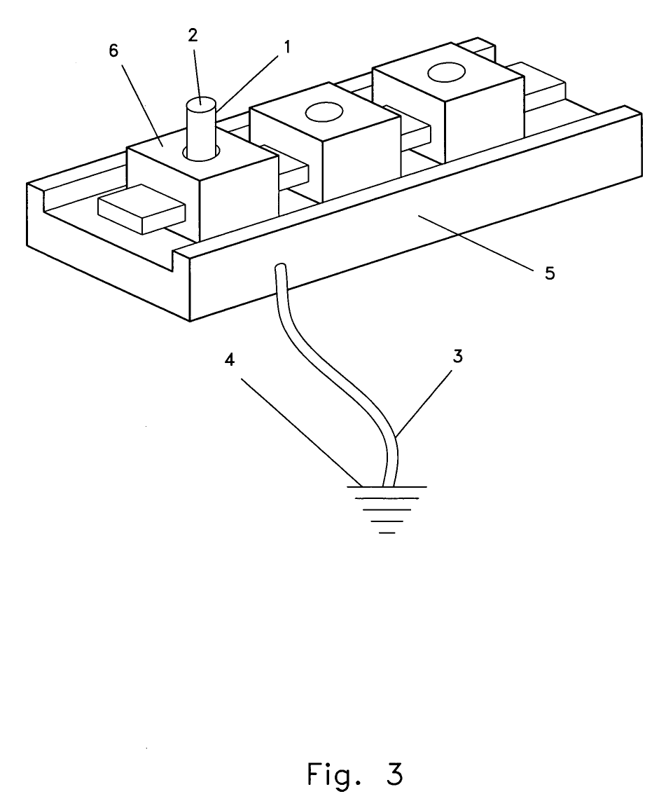

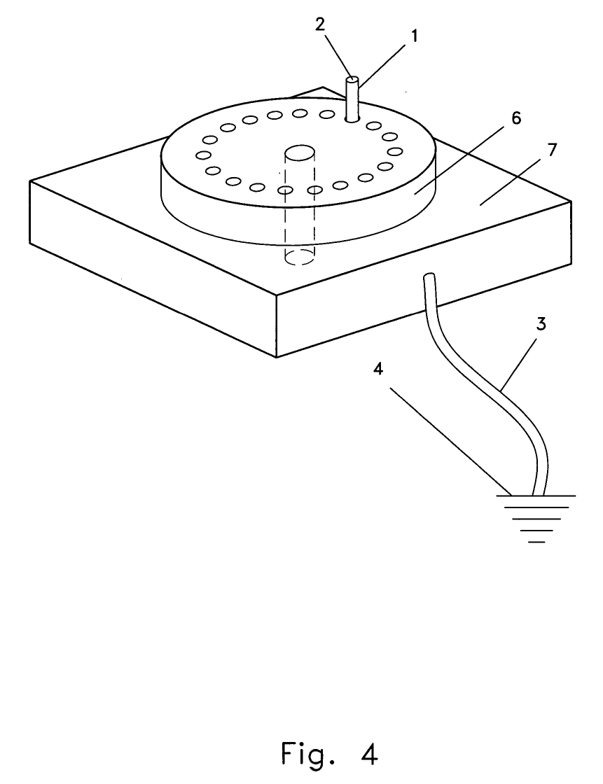

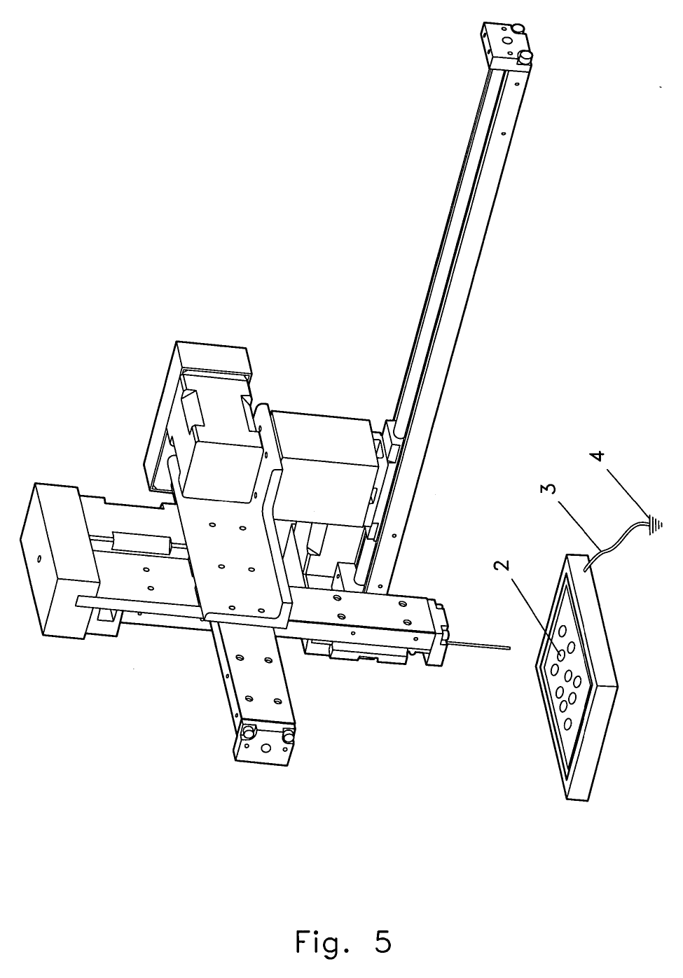

Now referring primarily to FIGS. 3, 4, and 5 the invention can further comprise a container body securement element (6) that holds the container body (1) in a substantially fixed orientation or a plurality of container bodies (1) in a substantially fixed orientation. With respect to certain embodiments of the invention, the container body securement element can comprise a test tube holder, microtiter plate holder (as shown in FIG. 5), the carousel of a fraction collector (as shown in FIG. 4), the interlocking segments of flexible track in a conveyer (as shown in FIG. 3), as but a few examples. These components can further comprise an amount of electrically conductive material impregnated throughout the container body securement element sufficient to allow the securement element (6) to maintain neutral charge when electrically connected to ground.

The invention can further comprise a container body securement element movement means (7) to position at least one said container body at a location. The securement element movement means can, for example, be a electric motor that indexes or turns a carousel, an XY axis that positions a welled tray, or the like.

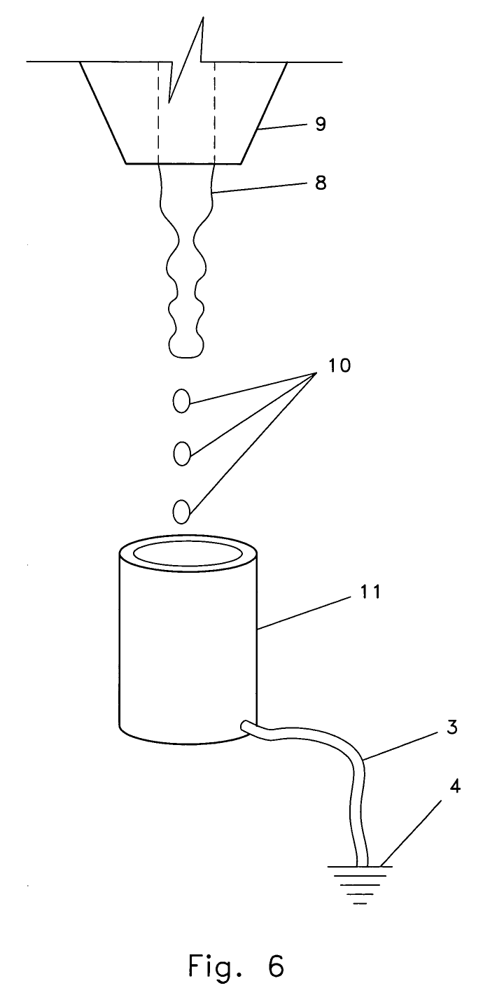

Now referring primarily to FIG. 6, the invention can comprise a droplet collector. The droplet collector can include a liquid stream (8) a droplet formation element (9) responsive to the liquid stream that causes the formation of a plurality of droplets (10) and a droplet collection element (11) having a charge dissipation element, such as the ground connection (3), as shown. The droplet formation element may be associated with a variety of instruments such as flow cytometers, liquid chromatographs, or the like. As discussed above, droplets charged with the same electrical polarity can be accumulated in the droplet collection element, the net result can be that the droplet collection element itself assumes the electrical polarity of the droplets being collected. Once this electrical charge of the droplet collector becomes established, incoming droplets of the same polarity can be deflected away from the droplet collection element since there is an electrostatic force operating to separate like charges. As such, an electrically charged droplet collection element can cause the loss of particles that normally would be collected in the collection container. The droplet collection element (11) can have a variety of configurations, including those discussed above, and have a sufficient amount of electrically conductive material impregnated throughout so that the droplet collection container itself can conduct electricity or move electrons as discussed above.

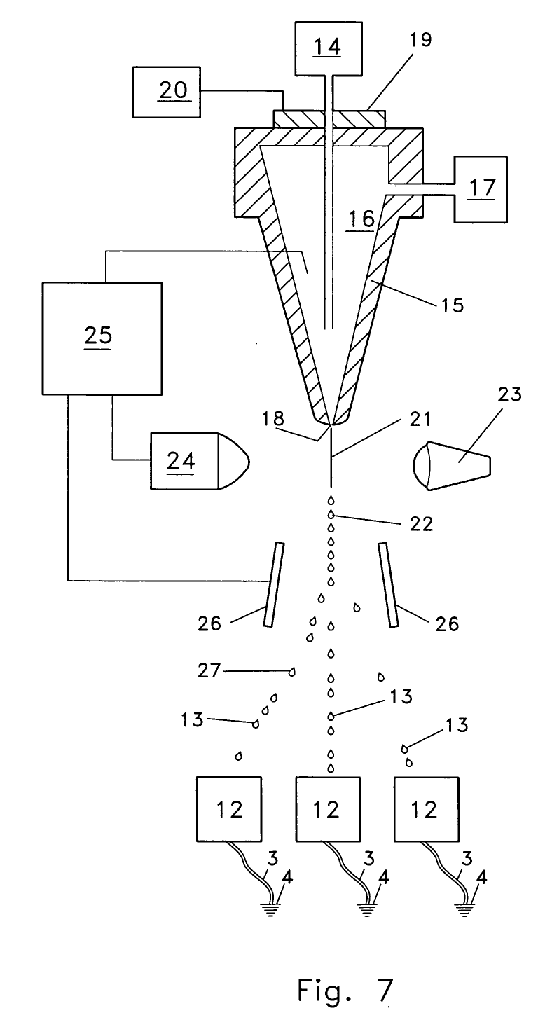

Now referring to primarily to FIG. 7, an embodiment of the invention can comprise a flow cytometer having electrically conductive collection containers (12) to enhance the collection and retention of electrostatically charged particles (13). Essentially, a flow cytometry involves the sorting items, such as cells, which are provided to the flow cytometer instrument through some type of cell source. A conceptual instrument is shown in FIG. 7. The flow cytometer instrument includes a cell source (14) that acts to establish or supply cells or some other type of item to be analyzed by the flow cytometer. The cells are deposited within a nozzle (15) in a manner such that the cells are surrounded by a sheath fluid (16). The sheath fluid (16) is usually supplied by some sheath fluid source (17) so that as the cell source (14) supplies its cells, the sheath fluid (16) is concurrently fed through the nozzle (18). In this manner it can be easily understood how the sheath fluid (16) forms a sheath fluid environment for the cells. Since the various fluids are provided to the flow cytometer at some pressure, they flow out of nozzle (15) and exit at the nozzle orifice (18). By providing some type of oscillator (19) which may be very precisely controlled through an oscillator control (20), pressure waves may be established within the nozzle (15) and transmitted to the fluids exiting the nozzle (15) at nozzle orifice (18). Since the oscillator (20) thus acts upon the sheath fluid (16), the stream (21) exiting the nozzle orifice (18) eventually and regularly forms drops (22). Because the cells are surrounded by a sheath fluid environment, the drops (22) may contain within them individually isolated (generally) cells or other items.

Since the drops (22) generally contain isolated cells or particles, the flow cytometer can distinguish and separate droplets based upon whether or not the appropriate cell(s) or particle(s) is/are contained within the droplet (22). This is accomplished through a cell sensing system. The cell sensing system involves at least some type of sensor (23) which responds to the cells contained within each droplet (22) as discussed in U.S. Pat. No. 5,135,759, hereby incorporated by reference herein. The cell sensing system may cause an action depending upon the relative presence or relative absence of a particular cell characteristic, for example the magnitude of fluorescence of a flurochrome bound to a cell or component of a cell. Thus in the example of a fluorochrome, by sensing magnitude of fluorescence upon excitation by an exciter element (23), such as a laser, associated with a particular cell it is possible to discriminate between the cells or other particles by their differing emission levels.

In order to achieve the ultimate separation and isolation of the appropriate cells, the signals received by sensor (24) are fed to some type of sorter discrimination system (25) that very rapidly analyzes the characteristics of the particle(s) entrained in the drop (22) and can differentially charge each drop (22) based upon whether it has decided that the desired cell or particle does or does not exist within that drop (22). In this manner the sorter discrimination system acts to permit the electrostatic deflection plates (26) to deflect drops (13) based on whether or not they contain the appropriate cell or other item. As a result, the flow cytometer acts to sort the cells or particles by causing them to land in one or more electrically neutral droplet collector(s) (12) of the various types or configurations described above or equivalents thereof. Thus by sensing some property of the cells or other items the flow cytometer can discriminate between cells based on a particular characteristic and place them in the appropriate electrically neutral droplet collector (12). With respect to typical flow cytometers used to differentiate particles or cells (27), the particles can be charged positively and thus deflect in one direction, the particles can be charged negatively and thus deflect the other way, and the wasted stream that remains uncharged and thus is collected in an undeflected stream into a suction tube or the like.

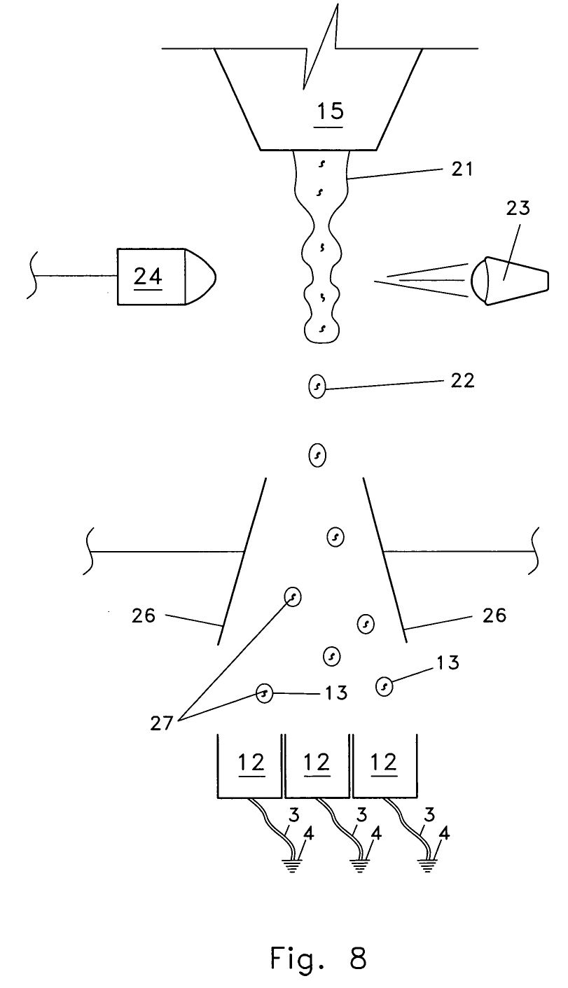

Now referring primarily to FIG. 8, the process can be even further understood. As shown in that figure, the nozzle (15) emits a stream (21) which because of the oscillator (19) (not shown in FIG. 8) forms drops (22). Since the cell source (14) (not shown in FIG. 2) may supply cells or particles (27) which may be stained, the light stimulation by laser exciter (23) is differentially determined by sensor (24) so that the existence or nonexistence of a charge on each drop (22) as it separates from stream (21) can be controlled by the flow cytometer. This control results in deflected droplets (13) positively charged, negatively charged, or uncharged drops, based upon their content.. These deflected drops (13) are those containing cells or particles (27) of the one or the other sex. They are then deposited in the appropriate electrically conductive/neutral droplet collector (12) for later use. The electrically neutral droplet collector can be responsive to a securement element (6), such as those described above for example, or movement means such as those described above.

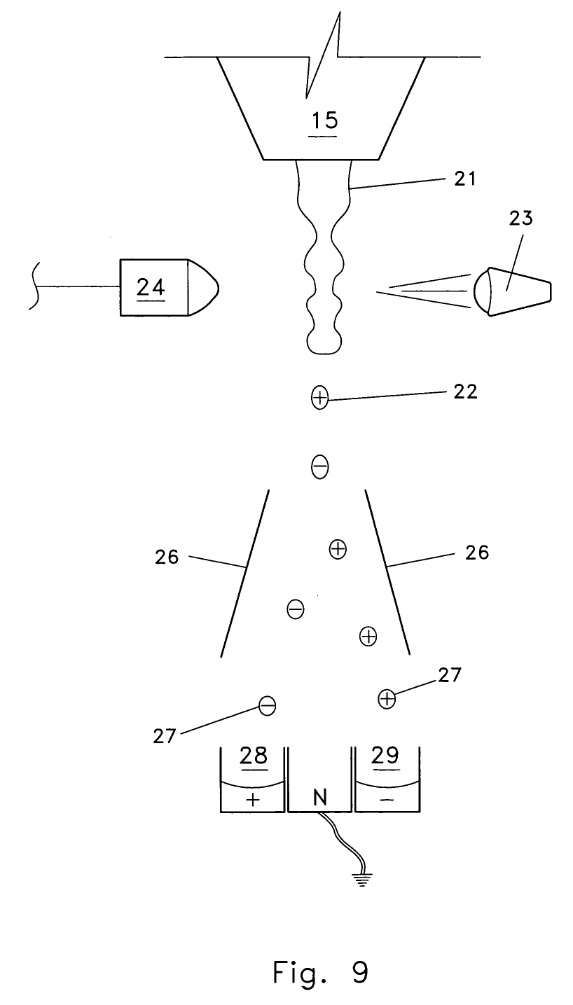

Now referring to FIG. 9, embodiments of the invention can comprise electrically conductive droplet collection containment elements (28) (29) that are charged with a desired positive or desired negative charge to attract particles or droplets (27) that have the opposite charge. With respect to some embodiments of the invention, a portion of the collection containment element (which could be any of the various configurations described above, other similar configurations, or equivalents thereof) could be selectively charged such as just the bottom portion of the collection containment element, as desired, or to accommodate a particular application based on the size, speed, trajectory, number, or total charge on a particles themselves. As described above, non-electrically conductive materials can be impregnated with electrically conductive polymers, or coated with a conductive coating to make the desired portion of the containment element conduct electricity so that it can maintain a desired magnitude of charge, either positive or negative, in the desired area of the containment element.

For example, in some embodiments of the invention a portion of the inside surface area of the containment element can coated with a thin film of conductive material by vacuum vapor distillation, sputtering, electrophoretic, or other methods of metal deposition. Alternately, the containment element when formed, or molded, or extruded, or the like can have the electrically conductive polymers allocated only the desired areas. The conductive areas of the containment elements can then be charged individually or in common in a charging circuit that imparts the desired type and magnitude of charge to the conductive portion of the containment element.

As can be easily understood from the foregoing, the basic concepts of the present invention may be embodied in a variety of ways. It involves various embodiments of electrically conductive or electrically neutralized containers for containment or collection of materials, such as droplets. In this patent application, the methods and techniques used with the electrically conductive or electrically neutralized containers are disclosed as part of the results shown to be achieved by the various devices described and as steps that are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this international Patent Cooperation Treaty patent application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in functionally-oriented terminology, each aspect of the function can accomplished by a device, subroutine, or program. Apparatus claims may not only be included for the devices described, but also method or process claims may be included to address the functions the invention and each element performs. Neither the description nor the terminology is intended to limit the scope of the claims.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “container”should be understood to encompass disclosure of the act of “containing”—whether explicitly discussed or not—and, conversely, were there only disclosure of the act of “containing”, such a disclosure should be understood to encompass disclosure of a “container” and even a “means for containing”. Such changes and alternative terms are to be understood to be explicitly included in the description.

Additionally, the various combinations and permutations of all elements or applications can be created and presented. All can be done to optimize the design or performance in a specific application.

Any acts of law, statutes, regulations, or rules mentioned in this application for patent; or patents, publications, or other references mentioned in this application for patent, are each hereby incorporated by reference. Specifically, U.S. Provisional Patent Application No. 60/256,070, filed Dec. 15, 2000, is hereby incorporated by reference including any figures or attachments, and each of the references in the following table of references are hereby incorporated by reference.

| | Issue | | | | |
|---|---|---|---|---|---|
| Pat. No. | Date | Name | Class | Subclass | Filing Date |
| 3,299,354 | Dec. 17, 1967 | Hogg | | | Jul. 5, 1962 |
| 3,661,460 | May 9, 1972 | Elking et al. | | | Aug. 28, 1970 |
| 3,710,933 | Jan. 16, 1973 | Fulwyler et al | | | Dec. 23, 1971 |
| 3,761,941 | Sep. 25, 1973 | Robertson | | | Oct. 13, 1972 |
| 3,810,010 | May 7, 1974 | Thom | | | Nov. 27, 1972 |
| 3,826,364 | Jul. 30, 1974 | Bonner et al | | | May 22, 1972 |
| 3,833,796 | Nov. 3, 1974 | Fetner et al | | | Oct. 13, 1971 |
| 3,960,449 | Jul. 1, 1976 | Carleton et al | | | Jun. 5, 1975 |
| 3,963,606 | Jun. 15, 1976 | Hogg | | | Jun. 3, 1974 |
| 3,973,196 | Aug. 3, 1976 | Hogg | | | Jun. 5, 1975 |
| 4,014,611 | Mar. 29, 1977 | Simpson et al | | | Apr. 30, 1975 |
| 4,070,617 | Jan. 24, 1978 | Kachel et al | | | Aug. 3, 1976 |
| 4,162,282 | Jul. 24, 1979 | Fulwyler et al | | | Apr. 22, 1976 |
| 4,230,558 | Oct. 28, 1980 | Fulwyler | | | Oct. 2, 1978 |
| 4,302,166 | Nov. 24, 1981 | Fulwyler et al | | | Mar. 15, 1979 |
| 4,317,520 | Mar. 2, 1982 | Lombardo et al | | | Aug. 20, 1979 |
| 4,318,480 | Mar. 9, 1982 | Lombardo et al | | | Aug. 20, 1979 |
| 4,318,481 | Mar. 9, 1982 | Lombardo et al | | | Aug. 20, 1979 |
| 4,318,482 | Mar. 9, 1982 | Barry et al | | | Aug. 20, 1979 |
| 4,325,483 | Apr. 20, 1982 | Lombardo et al | | | Aug. 20, 1979 |
| 4,341,471 | Jul. 27, 1982 | Hogg et al | | | Jan. 2, 1979 |
| 4,350,410 | Sep. 21, 1982 | Minott | | | Oct. 8, 1980 |
| 4,361,400 | Nov. 30, 1982 | Gray et al | | | Nov. 26, 1980 |
| 4,395,397 | Jun. 6, 2005 | Shapiro | | | |
| 4,395,676 | Jul. 26, 1983 | Hollinger et al | | | Nov. 24, 1980 |
| 4,400,764 | Aug. 23, 1983 | Kenyon | | | May 19, 1981 |
| 4,487,320 | Dec. 11, 1984 | Auer | | | Nov. 3, 1980 |
| 4,498,766 | Feb. 12, 1985 | Unterleitner | | | Mar. 25, 1982 |
| 4,515,274 | May 7, 1985 | Hollinger et al | | | Dec. 2, 1981 |
| 4,523,809 | Jun. 18, 1985 | Toboada et al | | | Aug. 4, 1983 |
| 4,538,733 | Nov. 3, 1985 | Hoffman | | | Oct. 14, 1983 |
| 4,598,408 | Jul. 1, 1986 | O'Keefe | | | 0/22/84 |
| 4,600,302 | Jul. 15, 1986 | Sage, Jr. | | | Mar. 26, 1984 |
| 4,631,483 | Dec. 23, 1986 | Proni et al | | | Feb. 1, 1984 |
| 4,673,288 | Jun. 16, 1987 | Thomas et al | | | Nov. 7, 1984 |
| 4,691,829 | Sep. 8, 1987 | Auer | | | Dec. 6, 1984 |
| 4,702,598 | Oct. 27, 1987 | Bohmer | | | Feb. 25, 1985 |
| 4,744,090 | May 10, 1988 | Freiberg | | | Jul. 8, 1985 |
| 4,756,427 | Jun. 11, 2005 | Schumann | | | |
| 4,758,729 | Jul. 19, 1988 | Monnin | | | Aug. 28, 1987 |
| 4,794,086 | Jan. 27, 1988 | Kasper et al | | | Nov. 25, 1985 |
| 4,818,103 | Apr. 4, 1989 | Thomas et al | | | Jan. 20, 1987 |
| 4,831,385 | May 16, 1989 | Archer et al | | | Oct. 14, 1987 |
| 4,845,025 | Jul. 4, 1989 | Lary et al | | | Nov. 10, 1987 |
| 4,877,965 | Oct. 31, 1989 | Dandliker et al | 250 | 458.1 | Jul. 1, 1985 |
| 4,942,305 | Jul. 17, 1990 | Sommer | | | May 12, 1989 |
| 4,981,580 | Jan. 1, 1991 | Auer | | | May 1, 1989 |
| 4,983,038 | Jan. 8, 1991 | Ohki et al | | | Apr. 7, 1988 |
| 5,005,981 | Apr. 9, 1991 | Schulte et al | | | Sep. 8, 1989 |
| 5,007,732 | Apr. 16, 1991 | Ohki et al | | | Apr. 18, 1988 |
| 5,030,002 | Jul. 9, 1991 | North, Jr. | | | Aug. 11, 1989 |
| 5,034,613 | Jul. 23, 1991 | Denk et al | 250 | 458.1 | Nov. 14, 1989 |
| 5,079,959 | Jan. 14, 1992 | Miyake et al | | | Sep. 8, 1989 |
| 5,098,657 | Mar. 24, 1992 | Blackford et al | | | Aug. 7, 1989 |
| 5,101,978 | Apr. 7, 1992 | Marcus | | | Nov. 27, 1989 |
| 5,127,729 | Jul. 7, 1992 | Oetliker et al | | | Oct. 15, 1986 |
| 5,144,224 | Sep. 1, 1992 | Larsen | | | Apr. 1, 1991 |
| 5,150,313 | Sep. 22, 1992 | Van den Engh et al | | | Apr. 12, 1990 |
| 5,159,397 | Oct. 27, 1992 | Kosaka et al | | | Sep. 5, 1991 |
| 5,159,403 | Oct. 27, 1992 | Kosaka | | | Mar. 19, 1991 |
| 5,167,926 | Dec. 1, 1992 | Kimura et al | | | Sep. 11, 1990 |
| 5,180,065 | Jan. 19, 1993 | Touge et al | | | Oct. 11, 1990 |
| 5,182,617 | Jan. 26, 1993 | Yoneyama et al | | | Jun. 29, 1990 |
| 5,199,576 | Apr. 6, 1993 | Corio et al | | | Apr. 5, 1991 |
| 5,215,376 | 06/93 | Schulte et al. | | | |
| 5,247,339 | Sep. 21, 1993 | Ogino | | | Sep. 5, 1991 |
| 5,259,593 | Nov. 9, 1993 | Orme et al | | | Apr. 16, 1992 |
| 5,260,764 | Nov. 9, 1993 | Fukuda et al | | | May 29, 1990 |
| 5,298,967 | Mar. 29, 1994 | Wells | | | Jun. 2, 1992 |
| 5,359,907 | Nov. 1, 1994 | Baker et al | | | Nov. 12, 1992 |
| 5,370,842 | Dec. 6, 1994 | Miyazaki et al | | | Nov. 20, 1992 |
| 5,412,466 | May 2, 1995 | Ogino | | | May 22, 1992 |
| 5,452,054 | Sep. 19, 1995 | Dewa et al | | | Nov. 21, 1994 |
| 5,466,572 | Nov. 14, 1995 | Sasaki, et al | | | Apr. 25, 1994 |
| 5,464,581 | Nov. 7, 1995 | Van den Engh et al | 422 | 82.01 | Aug. 2, 1993 |
| 5,467,189 | Nov. 14, 1995 | Kreikebaum et al | | | Jan. 12, 1995 |
| 5,471,294 | Nov. 28, 1995 | Ogino | | | Sep. 29, 1994 |
| 5,483,469 | Jan. 9, 1996 | Van den Engh et al | | | Aug. 2, 1993 |
| 5,523,573 | Jun. 4, 1996 | Hanninen et al | 250 | 459.1 | Dec. 28, 1994 |
| 5,558,998 | Sep. 24, 1996 | Hammond, et al | | | Jun. 5, 1995 |
| 5,596,401 | Jan. 21, 1997 | Kusuzawa | | | Sep. 14, 1994 |
| 5,601,235 | Feb. 11, 1997 | Booker et al | | | Nov. 15, 1994 |
| 5,602,039 | Feb. 11, 1997 | Van Den Engh | | | Oct. 14, 1994 |
| 5,602,349 | Feb. 11, 1997 | Van Den Engh | | | Oct. 14, 1994 |
| 5,641,457 | Jul. 24, 1997 | Van den Engh et al | | | Apr. 25, 1995 |
| 5,643,796 | Jul. 1, 1997 | Van den Engh et al | | | Oct. 14, 1994 |
| 5,650,847 | Jul. 22, 1997 | Maltsev, et al | | | Jun. 14, 1995 |
| 5,672,880 | Sep. 30, 1997 | Kain | 250 | 458.1 | Mar. 15, 1996 |
| 5,675,401 | Oct. 7, 1997 | Wangler et al | | | Jun. 15, 1995 |
| 5,700,692 | Dec. 23, 1997 | Sweet | | | Sep. 27, 1994 |
| 5,707,808 | Jan. 13, 1998 | Roslaniec et al | | | Apr. 15, 1996 |
| 5,726,364 | Mar. 10, 1998 | Van den Engh | | | Feb. 10, 1997 |
| 5,759,767 | Jun. 2, 1998 | Lakowicz et al | 435 | 4 | Oct. 11, 1996 |
| 5,777,732 | Jun. 7, 1998 | Hanninen et al | 356 | 318 | Apr. 27, 1995 |
| 5,786,560 | Jul. 28, 1998 | Tatah et al | 219 | 121.77 | Jun. 13, 1997 |
| 5,796,112 | Aug. 18, 1998 | Ichie | 250 | 458.1 | Aug. 9, 1996 |
| 5,815,262 | Sep. 29, 1998 | Schrof et al | 356 | 318 | Aug. 21, 1996 |
| 5,824,269 | Oct. 20, 1998 | Kosaka et al | | | Dec. 22, 1995 |
| 5,835,262 | Nov. 10, 1998 | Iketaki et al | 359 | 352 | Dec. 28, 1995 |
| 5,912,257 | Jun. 15, 1999 | Prasad et al | 514 | 356 | Sep. 5, 1996 |
| 5,916,449 | Jun. 29, 1999 | Ellwart et al | | | Dec. 3, 1996 |
| 6,133,044 | Oct. 17, 2000 | Van den Engh | | | Apr. 16, 1996 |

| Patent | | |
|---|---|---|
| Number | Date | Country |
| DE 19549015 | Mar. 04, 1997 | Germany |
| EP 0160201 A2 | Mar. 21, 1985 | Europe |
| EP 0781985 A2 | Jul. 02, 1997 | Europe |
| EP 0025296 A2 | Aug. 19, 1980 | Europe |
| EP 0468100 A1 | Dec. 27, 1990 | Europe |
| EP 0160201 | Nov. 06, 1985 | Europe |
| EP 025296 A2 | Mar. 18, 1981 | Europe |
| EP 0468100 A1 | Dec. 27, 1990 | Europe |
| FR 2699678 A1 | Dec. 23, 1992 | France |
| JP 2024535 (A) | Jan. 26, 1990 | Japan |
| JP 4126064 | Apr. 27, 1992 | Japan |
| JP 4126064 (A) | 27/04/92 | Japan |
| JP 4126065 (A) | Apr. 27, 1992 | Japan |
| JP 4126066 (A) | Apr. 27, 1992 | Japan |
| JP 4126079 (A) | Apr. 27, 1992 | Japan |
| JP 4126080 (A) | Apr. 27, 1992 | Japan |
| JP 4126081(A) | Apr. 27, 1992 | Japan |
| JP 61139747 (A) | Jun. 27, 1986 | Japan (Abstract) |
| JP 61159135 (A) | Jul. 18, 1986 | Japan (Abstract) |
| SU1056008 | Nov. 23, 1983 | Soviet |
| SU1260778-A1 | Sep. 30, 1986 | Russia |
| WO 99/44037 | Sep. 02, 1999 | US |
| WO 96/12171 | Apr. 25, 1996 | US |
| WO 96/12172 | Apr. 25, 1996 | US |
| WO 96/12173 | Apr. 25, 1996 | US |

Asbury, C. L., et al., “Fluorescence Spectra of DNA Dyes Measured in a Flow Cytometer, Cytometry: 24 pp 234-242, 1996.

Axicon; Journal of the Optical Society of America; Vol. 44, #8, Eastman Kodak Company, Hawk-Eye Works, Rochester, N.Y., Sep. 10, 1953 pp.592-597

Bakker Schut, T. C., et al., “A new principle of cell sorting by using selective electroporation in a modified cell sorter”, Cytometry 11, 1990, pp 659-666.

Bigos, M., et al., “Nine Color Eleven Parameter Immunophenotyping using Three Laser Flow Cytometry”, Cytometry: 36, 1999, pp 36-45.

Denk, W., et al. (1995). Two-photon molecular excitation in laser scanning microscopy. Handbook of Biological Confocal Microscopy. J. B. Pawley, ed., Plenum Press, New York. pp 444-458.

Fuller, R. R. and Sweedler, J. V., “Characterizing Submicron Vesicles with Wavelength-Resolved Fluorescence in Flow Cytometry”, Cytometry: 25, 1996, pp 144-155.

Garner, D. L., et al.; “Quantification of the X- and Y- Chromosome-Bearing Spermatozoa of Domestic Animals by Flow Cytometry¹, Biology of Reproduction 28, pgs. 312-321, (1983)

Garner, D. L., et al., “Quantification of the X- and Y- Chromosome-Bearing Spermatozoa of Domestic Animals by Flow Cytometry □, Biology of Reproduction 28, pp. 312-321 (1983) Gauci, M. R., et al., “Observation of Single-Cell Fluorescence Spectra in Laser Flow”, Cytometry 25, 1996, pp 388-393.

Goppert-Mayer, M. 1931,.Uber Elementarakte mit zwei Quantensprüngen. Annalen der Physik, Pages 273-294

Gottlinger, C., et al., “Operation of a Flow Cytometer□, Flow Cytometry and Cell Sorting, pp. 7-23 (1982)

Herweijer, H., et al., “High speed photodamage cell selection using bromodeoxyuridine/Hoechst 33342 photosensitized cell killing”, Cytometry 9, 1988, pp143-149.

Herzenberg, L., et al., “Fluorescence-activated Cell Sorting”, Scientific American, 234(3), pp 108-117.

Horan, P. and Wheeless, Jr., L., “Quantitative Single Cell Analysis and Sorting”, Science, 198, pp 149-157, October 1977.

Johnathan Sharpe's thesis: “Sperm Sexing using Flow Cytometry,” Chptr. 3.5-3.5.8, 1997

Johnson, L. A., “Sex Preselection by Flow Cytometric Separation of X and Y Chromosome-bearing Sperm based on DNA Difference: a Review□, Reprod. Fertil. Dev., pp. 893-903 (1995)

Johnson, Lawrence A., “Sex Preselection by Flow Cytometric Separation of X and Y Chromosome-bearing Sperm Based on DNA Difference: a Review, Reprod. Fertil. Dev., 1995, 7, pgs. 893-903

Keij, J. F., “The ZAPPER: a flow cytometer for high-speed photodamage cell selection”, PhD thesis, 1994.

Kinoshita, S., et al., “Spectroscopic Properties of Fluorescein in Living Lymphocytes”, Cytometry: 8, 1987, pp 35-41.

Manni, Jeff, (1996). Two-Photon Excitation Expands The Capabilities of Laser-Scanning Microscopy, Biophotonics International, pp 44-52

Martin, J. C., and Jett, J. H., “Photodamage, a basis for super high speed cell selection”, Cytometry 2, 1981 pp 114.

McLeod, J., Eastman Kodak Company, Hawk-Eye Works, Rochester, N.Y., Journal of the Optical Society of America; Vol. 44, no.8, September 1953, pp. 592-597

Melamed, M. R., et al., Flow Cytometry and Sorting, 2^{nd} ed. New York; Wiley Liss, 1990.

Melamed, M. R., et al., □An Historical Review of the Development of Flow Cytometers and Sorters□, A Review for Cytometers and Sorters, pp. 3-9, (1979)

PCT Application PCT/US99/04183, entitled “Method and Apparatus for Flow Cytometry”, filed on Feb. 26, 1999

PCT Application PCT/US95/14624, entitled “System for Sensing Droplet Formation Time Delay in a Flow Cytometer”, filed on Oct. 13, 1995

PCT Application PCT/US95/13502, entitled “Flow Cytometer Jet Monitor System”, filed on Oct. 13, 1995

PCT Application PCT/US95/13308, entitled “High Speed Flow Cytometer Droplet Formation System”, filed on Oct. 13, 1995

Pinkel, D., “Flow Chambers and Sample Handling”, Flow Cytometry: Instrumentation and Data Analysis, pp. 77-128 (1985)

Piston, D. W., et al. (1995). Three-dimensionally resolved NAD(P)H cellular metabolic redox imaging of the in-situ cornea with two-photon excitation laser scanning microscopy. J OF MICROSCOPY 178:20-27

Piston, D. W., et al. (1994). Two-photon-excitation fluorescence imaging of three-dimensional calcium ion activity. APPLIED OPTICS 33:662-669

Radbruch (Ed.), A., Flow Cytometry and Cell Sorting, □Operation of a Flow Cytometer□ by Goettlinger et al., 1992, pp.7-23

Recktenwald, D., et al., Cell Separation Methods & Applications New York: Marcel Dekker Inc, 1998.

Shapiro, H., “Practical Flow Cytometry”, Alan R. Liss, Inc., 1985.

Shapiro, H. M., Practical Flow Cytometry (3 ed), Wiley-Liss, 1995.

Sharpe□s, Johnathan, thesis: “Sperm Sexing—Method of Johnson et al.”, Chptr. 3.6-4.3.4, 1997

Sharpe□s, Johnathan, thesis: “An Introduction of Flow Cytometry,”Chptr. 2-2.2, 1997

Sharpe□s, Johnathan, thesis: “Gender Preselection-Principle Scientific Options,” Chptr. 3.4-3.4.8, 1997

Skogen-Hagenson, M. J., et al., “A High Efficiency Flow Cytometer”, The Journal of Histochemistry and Cytochemistry, Vol. 25, No. 7, pp. 784-789 (1977)

“The Nobel Prize in Chemistry, 2000: Conductive polymers”, Kungl. cetenskapsakademien: The Royal Swedish Academy of Sciences, http://www.nobel.se/chemistrv/laureates/2000/press.html, 16 pages 2000

“The 2000 Nobel Prize in Chemistry”, Kungl. cetenskapsakademien: The Royal Swedish Academy of Sciences, http://www.nobel.se/chemistry/laureates/2000/press.html, 2 pages,2000

U.S. Application 09/032,733, entitled “Method and Apparatus for Flow Cytometry”, filed on Feb. 27, 1998

U.S. Application 60/205,008, entitled “Flow Cytometer with Active Automated Optical Alignment System”, filed on May 17, 2000

U.S. Application 08/323,270, entitled “Flow Cytometer with Active Automated Optical Alignment System”, filed on Oct. 14, 1994

U.S. Application 60/206,633, entitled “Multi-Line Wavelength Excitation Apparatus For Flow Cytometry”, filed on May 24, 2000

U.S. Application 60/205,730, entitled “A Rapid Multi-Material Sample Input System For a Flow Cytometer”, filed on May 19, 2000

U.S. Application 09/689,585, entitled “Flow Cytometer Droplet Formation System”, filed on Oct. 12, 2000

U.S. Application 60/205,988, entitled “Use of Electrically Conductive Receptacles to Enhance Retention of Electrostatically Deflected Material”, filed on Dec. 15, 2000

U.S. Provisional Application 60/256,070, entitled “Flow Cytometer With Spectral Radiance Measurement Apparatus”, filed on May 19, 2000

Van Dilla et al. (Eds.), Flow Cytometry: Instrumentation and Data Analysis, □Flow Chambers and Sample Handling,□ by Pinkel et al., 1985, pp.77-128

Van Dilla, M., “Overview of Flow Cytometry: Instrumentation and Data Analysis”, Flow Cytometry: Instrumentation and Data Analysis, pp. 1-8 (1985)

Williams, R. M. et al. (1944). Two photon molecular excitation provides intrinsic 3-dimensional resolution for laser-based microscopy and microphotochemistry. FASEB J. 8:804-813.

In addition, as to each term used it should be understood that unless its utilization in this application is inconsistent with such interpretation, common dictionary definitions should be understood as incorporated for each term and all definitions, alternative terms, and synonyms such as contained in the Random House Webster's Unabridged Dictionary, second edition are hereby incorporated by reference. However, as to each of the above, to the extent that such information or statements incorporated by reference might be considered inconsistent with the patenting of this/these invention(s) such statements are expressly not to be considered as made by the applicant(s).

In addition, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible in countries such as Australia and the like.

Thus, the applicant(s) should be understood to have support to claim at least: i) each of the electrically conductive containers or electrically neutralized containers as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, and x) the various combinations and permutations of each of the elements disclosed.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the subject matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

I claim:

1. A method of flow cytometry, comprising:
a. establishing a fluid stream;
b. entraining particles in said fluid stream;
c. perturbing said fluid stream;
d. forming droplets some of which contain said particles;
e. analyzing said particles contained by said droplets;
f. charging said droplets based upon analysis of at least one characteristic of said particles;
g. deflecting said droplets based upon electrical charge of said droplet;
h. dissipating electrical charge of a droplet collection element; and
i. collecting said droplets in said droplet collection element.

2. A method of flow cytometery as described in claim 1, wherein said step of dissipating electrical charge of a droplet collection element comprises connecting said droplet collection element to a ground.

3. A method of flow cytometery as described in claim 2, wherein said step of connecting said droplet collection element to ground comprises connecting said droplet collection element electrically to ground.

4. A method of flow cytometery as described in claim 3, further comprising the step of neutralizing charge on said droplet collection element.

5. A method of flow cytometery as described in claim 4, further comprising the step of securing said droplet collection element in a substantially fixed orientation.

6. A method of flow cytometery as described in claim 5, further comprising the step of securing a plurality of said droplet collection elements in a substantially fixed spatial geometry.

7. A method of flow cytometery as described in claim 6, further comprising the step of dissipating electrical charge on said plurality of said droplet collection elements in said substantially fixed spatial geometry.

8. A method of flow cytometery as described in claim 7, further comprising the step of neutralizing charge on said plurality of said droplet collection elements having a substantially fixed spatial geometry.

9. A method of flow cytometry as described in claims 1, further comprising the step of impregnating said droplet collection element with an electrically conducting material.

10. A method of flow cytometry as described in claim 9, wherein said step of impregnating said droplet collection element with an electrically conducting material comprises forming said droplet collection element with an amount of polymer along which electrons can be transferred.

11. A method of flow cytometry as described in claim 10, further comprising the step of doping said polymer.

12. A method of flow cytometry as described in claim 10, wherein said polymer comprises a carbon polymer.

13. A method of flow cytometry as described in claim 10, wherein said carbon polymer has alternating single and double bonds.

14. A material containment device, comprising:
a. a container body configured to have at least one material containment element;
b. an discrete amount of electrically conductive material impregnated throughout said container body sufficient to allow said container body to maintain neutral charge when connected to ground.

15. A material containment device as described in claim 12, wherein said container body configured to have at least one material containment element comprises a container body configured from a substantially non-electrically conducting material.

16. A material containment device as described in claim 12, wherein said container body configured to have at least one material containment element comprises a container body configured from a plastic material.

17. A material containment device as described in claim 12, wherein said plastic material is selected from the group consisting of polystyrene, polycarbonate, polypropylene, polyacrylate, and fluorocarbon.

18. A material containment device as described in claim 12, wherein said electrically conductive material impregnated throughout said container body is selected from the group consisting of a carbon polymer through which electrons can be transferred

19. A material containment device as described in claims 15, 16, 17, or 18, wherein said at least one material containment element comprises a plurality of containment elements configured in columns and rows.

20. A material containment device as described in claim 19, wherein said at least one material containment element comprises a welled tray having a number of wells selected from the group consisting of 12, 24, 48, and 96.

21. A material containment device as described in claims 15, 16, 17, or 18, wherein said at least one material containment element comprises a tube having one closed end.

22. A material containment device as described in claim 19, further comprising a container body securement element, wherein said container body securement element has a discrete amount of electrically conductive material impregnated throughout sufficient to allow said container body to maintain neutral charge when connected to ground.

23. A material containment device as described in claim 21, further comprising a container body securement element, wherein said container body securement element has a discrete amount of electrically conductive material impregnated throughout sufficient to allow said container body to maintain neutral charge when connected to ground.

24. A material containment device as described in claim 23, further comprising a container body securement element movement means to position at least one said container body at a location.

25. A droplet collector, comprising:
a. a liquid stream;
b. a droplet formation element responsive to said liquid stream;
c. a plurality of droplets formed by said droplet formation element;
d. a droplet collection element into which at least some of said droplets are collected; and
e. a charge dissipation element electrically coupled to said droplet collection element.

26. A droplet collector as described in claim 25, wherein said charge dissipation element comprises a ground connection.

27. A droplet collector as described in claim 26, wherein said ground connection comprises an electrical conductor coupled between said droplet collection element and ground.

28. A droplet collector as described in claim 25, wherein said droplet collection element comprises a substantially tubular configuration having one closed end.

29. A droplet collector as described in claim 25, wherein said droplet collection element comprises a plurality of said droplet collection elements having said substantially tubular configuration.

30. A droplet collector as described in claim 29, wherein said plurality of said droplet collection elements of substantially tubular configuration are geometrically arranged in columns and rows.

31. A droplet collector as described in claim 30, wherein said plurality of said droplet collection elements of substantially tubular configuration geometrically arranged in columns and rows have a number of droplet collection elements selected from the group consisting of 12, 24, 48, and 96.

32. A droplet collector as described in claim 25, wherein said droplet collection element is configured from a substantially non-electrically conducting material.

33. A droplet collector as described in claim 32, wherein said droplet collection element is configured from a plastic material.

34. A droplet collector as described in claim 33, wherein said plastic material is selected from the group consisting of polystyrene, polycarbonate, polypropylene, polyacrylate, and fluorocarbon.

35. A droplet collector as described in claims 32, 33, or 34, further comprising an electrically conductive material impregnated throughout said droplet collection element.

36. A droplet collector as described in claim 35, wherein said electrically conductive material comprises a carbon polymer through which electrons can be transferred.

37. A droplet collector as described in claim 36, wherein said carbon polymer has alternating single and double bonds.

38. A droplet collector as described in claim 37, further comprising a droplet collection element securement, wherein said droplet element securement has an amount of electrically conductive material impregnated throughout sufficient to allow said droplet collection element to maintain a neutral charge when connected to ground.

39. A droplet collector as described in claim 38, further comprising a container body securement element movement means to position at least one said container body at a location.

40. A droplet collector as described in claim 39, further comprising particles contained in at least some of said plurality of droplets.

41. A droplet collector as described in claim 40, wherein said particles are cells

42. A flow cytometer, comprising:
a. a fluid stream;
b. a plurality of particles having particle characteristics entrained in said fluid stream;
c. a nozzle having an aperture through which said fluid stream exits;
d. an oscillator having an oscillation frequency that generates droplets at said aperture of said nozzle as said fluid stream exits, wherein at least a portion of said droplets entrain one of said plurality of particles;
e. a particle characteristic analyzer;
f. a droplet charger operably responsive to said droplets, wherein said droplet charger imparts an electrical charge to each of said droplets based upon analysis of said particle characteristics;
g. a droplet deflector that deflects said droplets;
h. an electrically conductive droplet collector into which said deflected droplets are collected.

43. A flow cytometer as described in claim 42, further comprising a ground connection between said electrically conductive droplet collector and ground.

44. A flow cytometer as described in claim 42, wherein said droplet collector comprises a substantially tubular configuration having one closed end.

45. A flow cytometer as described in claim 42, wherein said droplet collector comprises a plurality of said substantially tubular configurations each having one closed end.

46. A flow cytometer as described in claim 45, wherein said plurality of said substantially tubular configurations each having one closed end are geometrically arranged in columns and rows.

47. A flow cytometer as described in claim 46, wherein said plurality of said substantially tubular configurations each having one closed end geometrically arranged in columns and rows have a number of droplet collection elements selected from the group consisting of 12, 24, 48, and 96.

48. A flow cytometer as described in claim 47, wherein said droplet collector is configured from a substantially non-electrically conducting material.

49. A flow cytometer as described in claim 48, wherein said droplet collector is configured from a plastic material.

50. A flow cytometer as described in claim 49, wherein said plastic material is selected from the group consisting of polystyrene, polycarbonate, polypropylene, polyacrylate, and fluorocarbon.

51. A flow cytometer as described in claims 42, 45, 46, 48, or 50, further comprising an amount of electrically conductive material impregnated throughout said droplet collector.

52. A flow cytometer as described in claim 51, wherein said amount of electrically conductive material impregnated throughout said droplet collector is sufficient to allow said droplet collector to conduct electricity.

53. A flow cytometer as described in claim 52, wherein said electrically conductive material comprises a carbon polymer through which electrons can be transferred.

54. A flow cytometer as described in claim 53, wherein said carbon polymer has alternating single and double bonds.

55. A flow cytometer as described in claim 54, wherein said carbon polymer having alternating single and double bonds is doped.

56. A flow cytometer as described in claim 52, wherein said droplet collector further comprises a securement element configured to mate with said substantially tubular configurations each having one closed end, and wherein said securement element has an amount of electrically conductive material impregnated throughout sufficient to allow said droplet collector to conduct electricity.

57. A droplet collector as described in claim 56, further comprising droplet collector movement means to position said substantially tubular configurations each having one closed end when mated to said securement element.

58. A flow cytometer as described in claim 42, wherein said plurality of particles are cells.

59. A flow cytometer as described in claim 42, wherein said electrically conductive droplet collector has a portion of a material containment element that has selectably adjustable charge.

60. A flow cytometer as described in claim 42, wherein said selectably adjustable charge on said portion of said material containment element has a charge opposite to the charge of the droplet collected in said material containment element.

61. Methods substantially as described hereinbefore and with reference to any of the accompanying examples.

62. Apparatuses substantially as described hereinbefore and with reference to any of the accompanying examples.
